(19) United States
(12) Patent Application Publication
(10) Pub. No.: US 2026/0245707 A1
(43) Pub. Date: Aug. 20, 2026

(54) Method and Apparatus for First Aid

(71) Applicant: Telefonaktiebolaget LM Ericsson (publ), Stockholm (SE)

(72) Inventor: Yanping Dang, Beijing (CN)

(21) Appl. No.: 19/162,304

(22) PCT Filed: Apr. 4, 2023
(86) PCT No.: PCT/CN2023/086115

§ 371 Date: Sep. 4, 2025

(51) Int. Cl. G16H 40/20; G16H 15/00; G16H 20/00; G16H 40/67; G16H 50/20; G16H 80/00

(52) U.S. Cl. CPC G16H 40/20; G16H 15/00; G16H 20/00; G16H 40/67; G16H 50/20; G16H 80/00

(57) ABSTRACT

A method and an apparatus for first aid are provided. The method may include: receiving an event report from a user device; and dispatching a first aider at least based on his position; wherein the event report comprises an indication of a health event of a user of the user device.

### TECHNICAL FIELD

The non-limiting and exemplary embodiments of the present disclosure generally relate to the technical field of information processing, and specifically to a method and an apparatus for first aid.

### BACKGROUND

This section introduces aspects that may facilitate a better understanding of the disclosure. Accordingly, the statements of this section are to be read in this light and are not to be understood as admissions about what is in the prior art or what is not in the prior art.

With the development of personal health care hardware and software applications, people are enjoying more and more convenience of health care services. For example, a smart watch may monitor a heart rate of a user of the smart watch and a corresponding software may provide a report, a suggestion or an alarm to the user or the user's emergency contact.

When a user has an incident, e.g., falling down or heart attack, the smart watch may send a short message to the user's emergency contact or call public emergency number (911 in USA or 120 in China, etc.) and may share the user's position with the user's emergency contact or a public emergency center.

However, even with this advanced technology, the user still needs to wait for the public emergency center to dispatch emergency vehicles (on which there are doctors and nurses) to help the user. In crowded cities, it takes 30 mins to even hours for the emergency vehicles to arrive. This is not quick enough for most urgent matters.

Some organizations may train some employees to be first aiders for emergency help before the emergency vehicles arrive. However, the trained employees are listed in computer or on paper. People need to find the list and call trained employees one by one to see whether they are available nearby. This is also time-consuming.

There is a need to develop a technology capable of dispatching a first aider nearby quickly and accurately to provide medical care to the user who needs help.

### SUMMARY

This summary is provided to introduce a selection of concepts in a simplified form that are further described below in the detailed description. This summary is not intended to identify key features or essential features of the claimed subject matter, nor is it intended to be used to limit the scope of the claimed subject matter.

To overcome or mitigate at least one of the above mentioned problems or other problems or provide a useful solution, embodiments of the present disclosure propose a method and an apparatus for dispatching a first aider.

In a first aspect of the disclosure, there is provided a method for a first aid server. The method comprises: receiving an event report from a user device; and dispatching a first aider at least based on his position; wherein the event report comprises an indication of a health event of a user of the user device.

In an embodiment, the dispatching step comprises: receiving positions of multiple first aiders from their first aider devices; and selecting the first aider from the multiple first aiders at least based on their positions.

In an embodiment, the dispatching step further comprises: receiving capabilities and/or availabilities of the multiple first aiders from their first aider devices; and wherein the selecting is further based on their capabilities and/or availabilities.

In an embodiment, the method further comprises: establishing a voice call with the user device; and instructing a bystander how to help the user or gathering on-site information from the bystander through the voice call.

In an embodiment, the event report further comprises at least one of: age, height, weight, blood group, anamnesis, allergy, drug history and organ donation of the user.

In an embodiment, the dispatching step further comprises: sending a dispatch request to the first aider device of the first aider; and wherein the dispatch request comprises the event report.

In an embodiment, the dispatching step further comprises: navigating the first aider to the position of the user of the user device and/or a medical equipment/supply position.

In an embodiment, the method further comprises: establishing a voice call between the user device and the first aider device.

In an embodiment, the method further comprises: informing an emergency response center or an emergency contact of the user of the user device of the event report.

In a second aspect of the disclosure, there is provided a method for a user device. The method comprises: detecting a health event of a user of the user device; and sending an event report to a first aid server; wherein the event report comprises an indication of the health event of the user of the user device.

In an embodiment, the method further comprises: establishing a voice call with the first aid server; and wherein instruction to a bystander on how to help the user or on-site information from the bystander is transferred through the voice call.

In an embodiment, the method further comprises: collecting input from the user about at least one of: age, height, weight, blood group, anamnesis, allergy, drug history and organ donation of the user; and wherein the event report further comprises the collected input.

In an embodiment, the method further comprises: establishing a voice call with a first aider device via the first aid server.

In a third aspect of the disclosure, there is provided a method for a first aider device. The method comprises: sending a position report of the first aider device to a first aid server; and receiving a dispatch request from the first aid server; wherein the dispatch request comprises an indication of a health event of a user of a user device.

In an embodiment, the method further comprises: sending capabilities and/or availabilities of the first aider to the first aid server.

In an embodiment, the dispatch request further comprises at least one of: age, height, weight, blood group, anamnesis, allergy, drug history and organ donation of the user.

In an embodiment, the method further comprises: receiving navigation to the position of the user of the user device and/or a medical equipment/supply position.

In an embodiment, the method further comprises: establishing a voice call with the user device via the first aid server.

In a fourth aspect of the disclosure, there is provided an apparatus for. The apparatus includes: a communication interface; a processor; and a memory coupled to the processor, said memory containing instructions executable by said processor, whereby the apparatus is operative to perform a method according to any one of the first aspect, the second aspect and the third aspect.

In a fifth aspect of the disclosure, there is provided a computer-readable storage medium storing instructions which when executed by at least one processor, cause the at least one processor to perform the method according to the first aspect, the second aspect and the third aspect.

With the present invention, the user who needs medical help can get a first aider's help quickly.

### BRIEF DESCRIPTION OF THE DRAWINGS

The above and other aspects, features, and benefits of various embodiments of the present disclosure will become more fully apparent, by way of example, from the following detailed description with reference to the accompanying drawings, in which like reference numerals or letters are used to designate like or equivalent elements. The drawings are illustrated for facilitating better understanding of the embodiments of the disclosure and not necessarily drawn to scale, in which:

FIG. 1 shows a flowchart of a method 100 for a first aid server according to an embodiment of the present disclosure;

FIG. 2 shows a flowchart of a method 200 for a user device according to an embodiment of the present disclosure;

FIG. 3 shows a flowchart of a method 300 for a first aider device according to an embodiment of the present disclosure; and

FIG. 4 is a block diagram of an apparatus 400 for first aid according to embodiments of the present disclosure.

### DETAILED DESCRIPTION

The embodiments of the present disclosure are described in detail with reference to the accompanying drawings. It should be understood that these embodiments are discussed only for the purpose of enabling those skilled persons in the art to better understand and thus implement the present disclosure, rather than suggesting any limitations on the scope of the present disclosure. Reference throughout this specification to features, advantages, or similar language does not imply that all of the features and advantages that may be realized with the present disclosure should be or are in any single embodiment of the disclosure. Rather, language referring to the features and advantages is understood to mean that a specific feature, advantage, or characteristic described in connection with an embodiment is included in at least one embodiment of the present disclosure. Furthermore, the described features, advantages, and characteristics of the disclosure may be combined in any suitable manner in one or more embodiments. One skilled in the relevant art will recognize that the disclosure may be practiced without one or more of the specific features or advantages of a particular embodiment. In other instances, additional features and advantages may be recognized in certain embodiments that may not be present in all embodiments of the disclosure.

References in the specification to “one embodiment,” “an embodiment,” “an example embodiment,” and the like indicate that the embodiment described may include a particular feature, structure, or characteristic, but it is not necessary that every embodiment includes the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

It shall be understood that although the terms “first” and “second” etc. may be used herein to describe various elements, these elements should not be limited by these terms. These terms are only used to distinguish one element from another. For example, a first element could be termed a second element, and similarly, a second element could be termed a first element, without departing from the scope of example embodiments. As used herein, the term “and/or” includes any and all combinations of one or more of the associated listed terms.

The terminology used herein is for the purpose of describing particular embodiments only and is not intended to be limiting of example embodiments. As used herein, the singular forms “a”, “an” and “the” are intended to include the plural forms as well, unless the context clearly indicates otherwise. It will be further understood that the terms “comprises”, “comprising”, “has”, “having”, “includes” and/or “including”, when used herein, specify the presence of stated features, elements, and/or components etc., but do not preclude the presence or addition of one or more other features, elements, components and/or combinations thereof.

It is noted that the terms as used in this document are used only for ease of description and differentiation among nodes, devices or networks etc. With the development of the technology, other terms with the similar/same meanings may also be used.

In the following description and claims, unless defined otherwise, all technical and scientific terms used herein have the same meaning as commonly understood by one of ordinary skills in the art to which this disclosure belongs.

In the system of the present disclosure, there is a user device worn/carried by or attached to a user of the user device. The user device is capable of detecting various situation/status of the user and may report to a first aid server in communication network or in cloud. A first aider with a first aider device may be a trained volunteer, a registered doctor/nurse, someone from the red cross organization, a firefighter or others who can provide first aid/medical service to people. The first aid server may coordinate the first aiders via the first aider devices to help the user who needs medical help.

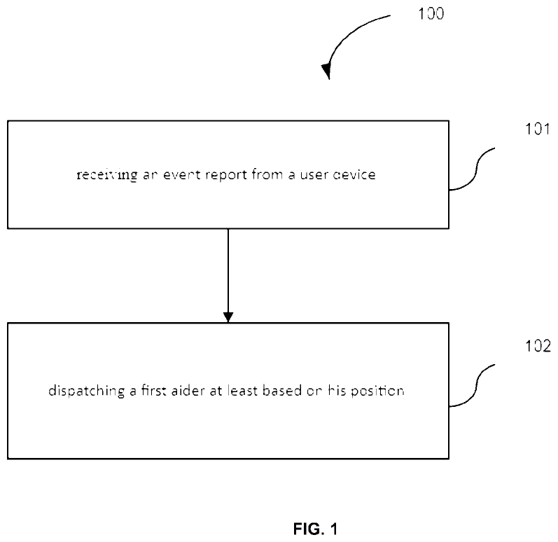

FIG. 1 shows a flowchart of a method 100 for a first aid server according to an embodiment of the present disclosure.

At block 101, an event report from a user device is received.

The first aid server may connect with the user device via various kinds of network. The user device may send an event report to the first aid server. The event report may comprise an indication of a health event of a user of the user device. The health event indicates that the user needs first aid/medical service, e.g., may indicate falling down, heart attack, low heart rate, low blood oxygen, losing consciousness, stop breathing, hypoglycemia, etc. In another embodiment, the event report may further comprise at least one of: age, height, weight, blood group, anamnesis, allergy, drug history and organ donation of the user. These information may be collected from the user and stored in the user device locally or in cloud and may be provided to the first aid server only when the user needs help so as to protect data safety/privacy.

At block 102, a first aider at least based on his position is dispatched.

The first aid server may collect the first aiders' information (e.g., name, capability, license, certificate, position, preference, language, etc.) in advance from the first aider devices, a database, a server, etc.

After receiving the event report from the user device, the first aider may dispatch a first aider to help the user of the user device. The dispatch action may be at least based on the first aider's position, because first aid is time sensitive. In one embodiment, the first aider with the smallest distance to the user is selected. In another embodiment, the first aider with a distance to the user within a preset threshold is selected. In yet another embodiment, one first aider or multiple first aiders may be selected.

The first aider's position may be acquired in advance, e.g., regularly, periodically, or randomly, regardless of receiving the event report. Alternatively, the first aid server may send a positioning report to the first aider devices after receiving the event report.

In yet another embodiment, the first aid server may receive capabilities and/or availabilities of first aiders from their first aider devices. As the above, this action may be performed in advance or in response to receiving the event report. The first aid server may dispatch (select) the first user(s) further based on their capabilities and/or availabilities. Capability shows how much/how well a first aider can provide medical care. For example, a professional first aid doctor can provide more/better help than an amateur volunteer. A cardiovascular doctor may be better than a dentist in helping a user with heart attack. Availability shows whether the first aider can provide help currently. The availability may be checked/confirmed with the first aider device.

After the dispatch decision is made, the first aid server may send a dispatch request to the first aider device of the selected first aider. The dispatch request may comprise the event report. The dispatch report may further comprise on-site information collected by the user device and sent to the first aid server.

In another embodiment, the user of the user device may be accompanied by a bystander who doesn't know how to help. Before the arrival of the first aider, it is useful for the bystander to provide information to the first aider or for the first aider to instruct the bystander what to do and what not to do. That is, the communication between the bystander and the first aider is beneficial for the user. Thus, the method 100 may further comprise: establishing a voice call between the user device and the first aider device. Moreover, the bystander normally does not know that a first aider can help and is on his/her way. With the present embodiment, the bystander can rest assured and/or help the user better with the user device of the user without having to carry a dedicated device and without need to unlock the user device of the user.

In another embodiment, the first aid server may establish a voice call with the user device and utilize Artificial Intelligence (AI) or Machine Learning (ML) to collect on-site information from the bystander, to instruct the bystander how to help the user, or to draw a bystander's attention by broadcasting a voice message and/or an alarm, through a voice call. This is very useful when the user himself/herself is unable to ask for help.

To help the first aider to arrive at the spot, in one embodiment, the dispatching step 102 may further comprise: navigating the first aider to the position of the user of the user device and/or a medical equipment/supply position. The medical equipment/supply may be a first aid supply station, a first aid kit, an Automated External Defibrillator, or the like.

In another embodiment, the method 100 may further comprise: informing an emergency response center or an emergency contact of the user of the user device of the event report. The emergency contact may be obtained from the user device.

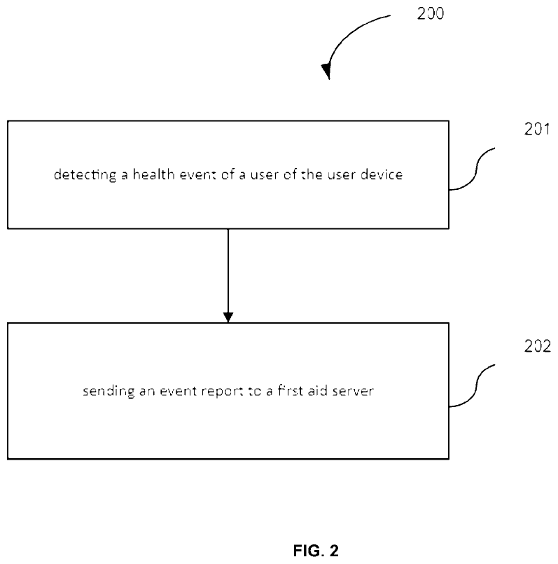

FIG. 2 shows a flowchart of a method 200 for a user device according to an embodiment of the present disclosure.

At block 201, a health event of a user of the user device is detected.

The user device may comprise, but not limited to: a mobile phone, a smart phone, a personal digital assistant, a computer, a wearable, a smart watch, a health monitor device, a smart ring, a smart tag, an oximeter, a smart bracelet, a smart necklace, smart glasses, a heart rate detector, etc.

The user device may be equipped with various sensors to enable the user device to sense the user's condition and may detect certain health related event of the user of the user device. For example, CN113936422A introduces how a mobile device can detect falling down of a user and the fact that the user needs help.

At block 202, an event report is sent to a first aid server.

In response to the detection of the health event, the user device may send the event report to the first aid server. The event report may comprise an indication of the health event of the user of the user device. As mentioned above, the health event indicates that the user needs first aid/medical service, e.g., may indicate falling down, heart attack, low heart rate, low blood oxygen, losing consciousness, stop breathing, hypoglycemia, etc. In another embodiment, the event report may further comprise at least one of: age, height, weight, blood group, anamnesis, allergy, drug history and organ donation of the user. These information may be collected from the user and stored in the user device locally or in cloud and may be provided to the first aid server only when the user needs help so as to protect data safety/privacy.

In an embodiment, the method 200 may further comprise: establishing a voice call with the first aid server. Instruction to a bystander on how to help the user or on-site information from the bystander may be transferred through the voice call.

In an embodiment, the method 200 may further comprise: establishing a voice call with a first aider device via the first aid server. In this scenario, the first aider and the bystander can communicate with each other to e.g., exchange information, navigation, instruct how to help the user, etc.

In an embodiment, the user device may broadcast a voice message or an alarm to ask for help from a bystander. This is very useful when the user himself/herself is unable to ask for help.

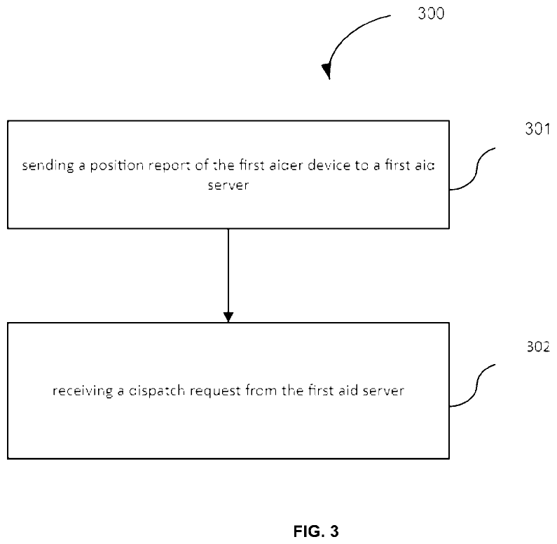

FIG. 3 shows a flowchart of a method 300 for a first aider device according to an embodiment of the present disclosure.

At block 301, a position report of the first aider device is sent to a first aid server.

As mentioned above, the position report may be sent regularly, periodically, randomly, or in response to a positioning request from the first aid server.

At block 302, a dispatch request from the first aid server is received.

The dispatch request comprises an indication of a health event of a user of a user device.

In one embodiment, the method 300 may further comprise: sending capabilities and/or availabilities of the first aider to the first aid server. The capabilities and/or availabilities may be sent before or after receiving the dispatch request.

In one embodiment, the dispatch request may further comprise at least one of: age, height, weight, blood group, anamnesis, allergy, drug history and organ donation of the user.

In one embodiment, the method 300 may further comprise: receiving navigation to the position of the user of the user device and/or a medical equipment/supply position.

In one embodiment, the method 300 may further comprise: establishing a voice call with the user device via the first aid server.

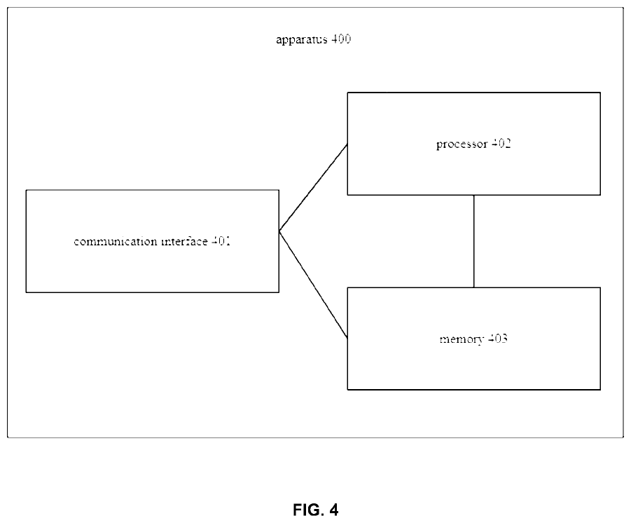

FIG. 4 is a block diagram of an apparatus 400 for first aid according to embodiments of the present disclosure.

The apparatus 400 includes a communication interface 401, a processor 402 and a memory 403. The memory 403 contains instructions executable by the processor 402 whereby the apparatus 400 is operative to perform the actions, e.g., of the procedure described earlier in conjunction with FIGS. 1-3.

In some embodiments, the memory 403 may further contain instructions executable by the processor 402 whereby the apparatus 400 is operative to perform any of the aforementioned methods, steps, and processes.

The present disclosure also provides at least one computer program product in the form of a non-volatile or volatile memory, e.g., a non-transitory computer readable storage medium, an Electrically Erasable Programmable Read-Only Memory (EEPROM), a flash memory and a hard drive. The computer program product includes a computer program. The computer program includes: code/computer readable instructions, which when executed by the processor 402 causes the apparatus 400 to perform the actions, e.g., of the procedure described earlier in conjunction with FIGS. 1-3.

The computer program product may be configured as a computer program code structured in computer program modules. The computer program modules could essentially perform the actions of the flow illustrated in FIGS. 1-3.

The processor may be a single CPU (Central processing unit), but could also comprise two or more processing units. For example, the processor may include general purpose microprocessors; instruction set processors and/or related chips sets and/or special purpose microprocessors such as Application Specific Integrated Circuit (ASICs). The processor may also comprise board memory for caching purposes. The computer program may be carried by a computer program product connected to the processor. The computer program product may comprise a non-transitory computer readable storage medium on which the computer program is stored. For example, the computer program product may be a flash memory, a Random-access memory (RAM), a Read-Only Memory (ROM), or an EEPROM, and the computer program modules described above could in alternative embodiments be distributed on different computer program products in the form of memories.

The techniques described herein may be implemented by various means so that an apparatus implementing one or more functions of a corresponding apparatus described with an embodiment comprises not only prior art means, but also means for implementing the one or more functions of the corresponding apparatus described with the embodiment and it may comprise separate means for each separate function or means that may be configured to perform two or more functions. For example, these techniques may be implemented in hardware (one or more apparatuses), firmware (one or more apparatuses), software (one or more modules), or combinations thereof. For a firmware or software, implementation may be made through modules (e.g., procedures, functions, and so on) that perform the functions described herein.

Exemplary embodiments herein have been described above with reference to block diagrams and flowchart illustrations of methods and apparatuses. It will be understood that each block of the block diagrams and flowchart illustrations, and combinations of blocks in the block diagrams and flowchart illustrations, respectively, can be implemented by various means including computer program instructions. These computer program instructions may be loaded onto a general purpose computer, special purpose computer, or other programmable data processing apparatus to produce a machine, such that the instructions which execute on the computer or other programmable data processing apparatus create means for implementing the functions specified in the flowchart block or blocks.

Further, while operations are depicted in a particular order, this should not be understood as requiring that such operations be performed in the particular order shown or in sequential order, or that all illustrated operations be performed, to achieve desirable results. In certain circumstances, multitasking and parallel processing may be advantageous. Likewise, while several specific implementation details are contained in the above discussions, these should not be construed as limitations on the scope of the subject matter described herein, but rather as descriptions of features that may be specific to particular embodiments. Certain features that are described in the context of separate embodiments may also be implemented in combination in a single embodiment. Conversely, various features that are described in the context of a single embodiment may also be implemented in multiple embodiments separately or in any suitable sub-combination.

While this specification contains many specific implementation details, these should not be construed as limitations on the scope of any implementation or of what may be claimed, but rather as descriptions of features that may be specific to particular embodiments of particular implementations. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

It will be obvious to a person skilled in the art that, as the technology advances, the inventive concept can be implemented in various ways. The above described embodiments are given for describing rather than limiting the disclosure, and it is to be understood that modifications and variations may be resorted to without departing from the spirit and scope of the disclosure as those skilled in the art readily understand. Such modifications and variations are considered to be within the scope of the disclosure and the appended claims. The protection scope of the disclosure is defined by the accompanying claims.

What is claimed is:

1. A method for a first aid server, comprising:
receiving an event report from a user device; and
dispatching a first aider at least based on his position;
wherein the event report comprises an indication of a health event of a user of the user device.

2. The method of claim 1, wherein the dispatching step comprises:
receiving positions of multiple first aiders from their first aider devices; and
selecting the first aider from the multiple first aiders at least based on their positions.

3. The method of claim 2, wherein the dispatching step further comprises:
receiving capabilities and/or availabilities of the multiple first aiders from their first aider devices; and
wherein the selecting is further based on their capabilities and/or availabilities.

4. The method of any of claims 1-3, further comprises:
establishing a voice call with the user device; and
instructing a bystander how to help the user or gathering on-site information from the bystander through the voice call.

5. The method of any of claims 1-4, wherein the event report further comprises at least one of: age, height, weight, blood group, anamnesis, allergy, drug history and organ donation of the user.

6. The method of any of claims 1-5, wherein the dispatching step further comprises:
sending a dispatch request to the first aider device of the first aider; and
wherein the dispatch request comprises the event report.

7. The method of any of claims 1-6, wherein the dispatching step further comprises:
navigating the first aider to the position of the user of the user device and/or a medical equipment/supply position.

8. The method of any of claims 1-7, further comprises:
establishing a voice call between the user device and the first aider device.

9. The method of any of claims 1-8, further comprises:
informing an emergency response center or an emergency contact of the user of the user device of the event report.

10. A method for a user device, comprising:
detecting a health event of a user of the user device; and
sending an event report to a first aid server;
wherein the event report comprises an indication of the health event of the user of the user device.

11. The method of claim 10, further comprises:
establishing a voice call with the first aid server; and
wherein instruction to a bystander on how to help the user or on-site information from the bystander is transferred through the voice call.

12. The method of any of claims 10-11, further comprises:
collecting input from the user about at least one of: age, height, weight, blood group, anamnesis, allergy, drug history and organ donation of the user; and
wherein the event report further comprises the collected input.

13. The method of any of claims 10-12, further comprises:
establishing a voice call with a first aider device via the first aid server.

14. A method for a first aider device, comprising:
sending a position report of the first aider device to a first aid server; and
receiving a dispatch request from the first aid server;
wherein the dispatch request comprises an indication of a health event of a user of a user device.

15. The method of claim 14, further comprises:
sending capabilities and/or availabilities of the first aider to the first aid server.

16. The method of any of claims 14-15, wherein the dispatch request further comprises at least one of: age, height, weight, blood group, anamnesis, allergy, drug history and organ donation of the user.

17. The method of any of claims 14-16, further comprises:
receiving navigation to the position of the user of the user device and/or a medical equipment/supply position.

18. The method of any of claims 14-17, further comprises:
establishing a voice call with the user device via the first aid server.

19. An apparatus for first aid, comprising:
a communication interface;
a processor; and
a memory coupled to the processor, said memory containing instructions executable by said processor, whereby the apparatus is operative to perform a method according to any one of claims 1-18.

20. A computer-readable storage medium storing instructions which when executed by at least one processor, cause the at least one processor to perform the method according to any one of claims 1-18.
